(19) United States
(12) Patent Application Publication
(10) Pub. No.: US 2019/0076610 A1
(43) Pub. Date: Mar. 14, 2019

(54) CPAP machine mask and hose holder

(71) Applicant: Joseph Stephen Alarid, Denver, CO (US)

(72) Inventor: Joseph Stephen Alarid, Denver, CO (US)

(21) Appl. No.: 15/704,660

(22) Filed: Sep. 14, 2017

(51) Int. Cl. A61M 16/06; A61M 16/08

(52) U.S. Cl. CPC A61M 16/06; A61M 2209/082; A61M 16/0875

(57) ABSTRACT

My invention came to me one morning when I woke up, took off my mask throw it at the desk missing, my dog came out from under the bed and bite into it. I tried to chase her, stepping on my mask cracking it. I decided to make a CPAP mask and hose holder. Something simple, made of plastic easy to snap the hose into, when not in use.

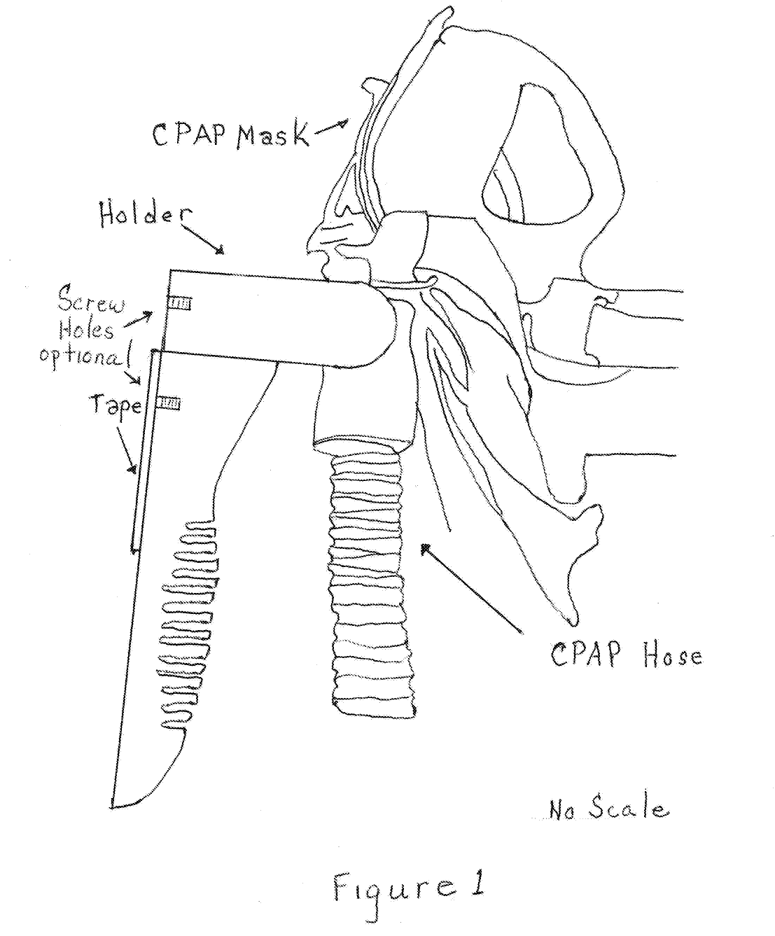

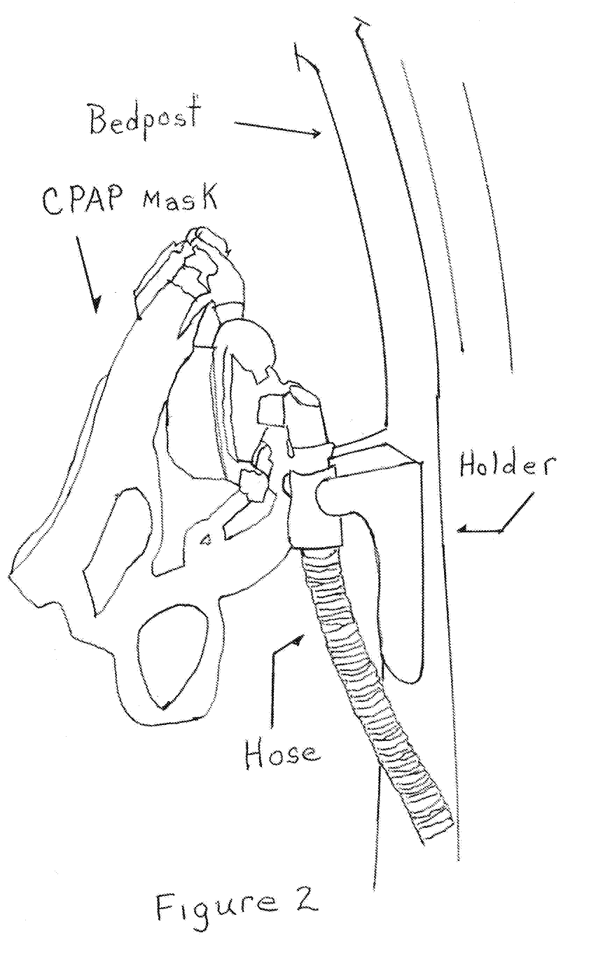

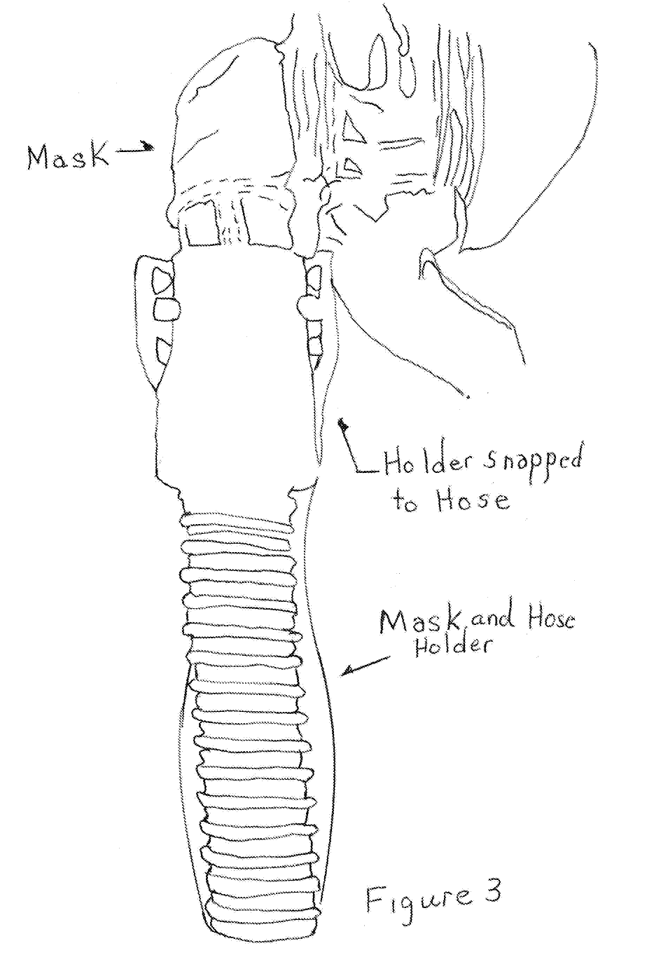

I thought if I made from glow bright plastic you could see it in the dark. I would make it easy to move. I believe the basic design is new and innovative. I could change the basic design for other medical hoses, it could be used on every hospital bed everywhere. The mask and hose snap works. Grab your mask and disconnect it from the holder ready for another good night of sleep. In the morning hang it in the holder safe it till you are ready to use it again.

I wanted to make a holder for the mask and hose I could attach anywhere I wanted to put it, and if you wanted to move it I could. The holder would have a handle that you can grab. A tape attached to the holder under the handle would remove the mask and hose from the holder. The mask and hose would then detach from the handle for cleaning and maintenance. The mask and hose would attach back to the handle and back to the holder.

A mask and hose holder for CPAP has not been made like this one. A process that no other provides by this method. I believe the basic design is new and innovative. I could modified the basic design for other medical hose holders. It could have a use on every bed in every hospital in America.

### 01. BACKGROUND OF THE INVENTION

A CPAP machine is a medical device used to provide continuous positive airway pressure. This is a treatment that uses mild air pressure to keep the airway open. CPAP machine are typically used by people who have breathing problems such as Sleep Apnea. There are more than 18 million people that could use the CPAP Mask and Hose Holder. There is no product in the market place that does what this invention will do.

### 02. BRIEF SUMMARY OF THE INVENTION

Most people place their mask and hose on a night stand or throw it aside, where it can be damaged or fall to the floor. On the floor it can be damaged by accidently being stepped on, or chewed on by a pet. The Mask and Hose Holder allows you snap them into it, where it hangs until the next night, when you need it again.

The CPAP Mask and Hose Holder is used to place your mask and hose in a safe place that is in arm reach when not in use. Your mask and hose snaps into the holder. Then attaches to the bedpost or night stand. This keeps the mask and hose from being damaged and out of the way. The mask and holder can be unattached wherever you install it and moved to any new location, then reattached and start using.

### 03. BRIEF DESCRIPTION OF THE DRAWING

No scale approximate size showing the handle holding the CPAP Hose snap into the holder. This CPAP hose is in common use with most machines but can be adaptive to any location of their choice.

Actual scale, the drawing shows the holder secured to the side of a common bed frame holding the hose and mask in a safe spot. No one makes a device like this one for that purpose. The handle can be removed so you repair, replace or clean the mask or hose, then put back in place.

No scale The CPAP hose is snapped into the holder with knob grabbers that are part of the plastic mold holder. The knob grabber size can be changed to fit different size hose and mask.

### 04. DETAILED DESCRIPTION OF THE INVENTION

The process for making the holder is a simple plastic mode. It consist of a handle 5″ in length and 1″ to snap the hose and mask into. The 6″ device is 1″ in wide and 2¾′ at the snap. 1″ at the center of the handle. Once you are done with the hose and mask for the night you have a place to store them. A safe clean spot, where it won't get damage, easy to remove for cleaning and maintenance. It also could be made of glow bright material (option) to make it easy to see in the dark. There is no invention like this to distinguish it to. It is a completely new method that makes a daily task for millions safer and easier to do. The plastic handle attaches to, tape or a screw for easy mounting.

The claims of the invention are:

1. The CPAP Mask and Hose Holder is new and innovative, no other device has ever been used in this way, for this purpose.

2. The Mask and Hose Holder could be used by over 18 million people with breathing problems that deal with a CPAP machine nightly.

3. Once you are done with the hose and mask snap into the holder, easy to unsnap for cleaning or maintenance or storage.
